(19) United States
(12) Patent Application Publication
(10) Pub. No.: US 2007/0224680 A1
(43) Pub. Date: Sep. 27, 2007

(54) VEHICLE TO TRANSPORT A DNA-MODIFYING ENZYME TO A GENOME

(75) Inventor: Marcel Herman Jozef Ruiters, Groningen (NL)

Correspondence Address: PEACOCK MYERS, P.C., 201 THIRD STREET, N.W., SUITE 1340, ALBUQUERQUE, NM, 87102, US

(73) Assignee: Synvolux IP B.V., Groningen (NL)

(21) Appl. No.: 11/738,393

(22) Filed: Apr. 20, 2007

Related U.S. Application Data
(63) Continuation-in-part of application No. PCT/NL05/00753, filed on Oct. 20, 2005.

(30) Foreign Application Priority Data
Oct. 21, 2004 (NL) 1027311
Nov. 4, 2004 (NL) 1027417
Nov. 10, 2004 (NL) 1027479

(51) Int. Cl. C12N 15/09

(52) U.S. Cl. 435/455

(57) ABSTRACT

A vehicle to transport a DNA-modifying enzyme towards a desired site in the genome, the DNA-modifying enzyme being coupled to a sequence recognizer that is specific for the desired site in the genome in such a way that the enzyme is able to perform its activity bound to the desired site.

### CROSS-REFERENCE TO RELATED APPLICATIONS

This application is a continuation-in-part utility application of Patent Cooperation Treaty (PCT) Ser. No. PCT/NL2005/000753, entitled “Vehicle to Transport a DNA-Modifying Enzyme to a Genome”, to Synvolux IP B.V., filed on Oct. 20, 2005, and the specification and claims thereof are incorporated herein by reference.

This application claims priority to and the benefit of the filing of Netherlands Patent Application Serial No. 1027311, entitled “Vehicle to Transport a DNA-Modifying Enzyme to a Genome”, filed on Oct. 21, 2004, and the specification and claims thereof are incorporated herein by reference.

This application claims priority to and the benefit of the filing of Netherlands Patent Application Serial No. 1027417, entitled “Vehicle for the Transport of a Chosen Molecule to a Cell”, filed on Nov. 4, 2004, and the specification and claims thereof are incorporated herein by reference.

This application claims priority to and the benefit of the filing of Netherlands Patent Application Serial No. 1027479, entitled “Protection of Biologically Active Molecules Using Amphiphiles”, filed on Nov. 10, 2004, and the specification and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BRIEF SUMMARY OF THE INVENTION

The present invention relates to a vehicle to transport a DNA-modifying enzyme to a desired site in a genome. Further, the invention relates to the application of the vehicle of the invention comprising a SAINT-molecule.

### DESCRIPTION OF RELATED ART

It is known from the state of the art that cancer and metabolic diseases are in many cases caused by undesired regulation of specific genes. In molecular biological research on these types of diseases, often DNA-modifying enzymes (such as restriction enzymes, methylases and demethylases, etc.) are used. These enzymes are specific in such a manner that, in the genome of humans, animals, plants, bacteria and viruses, they have a certain specificity for recognizing a particular nucleotide sequence. Specific nucleotide sequences of that sort are usually found several times, even sometimes thousands of times per genome.

The above described technology is known from the article by Paul S. Lovett and Michael G Bramucci, “Evidence of a non-random base sequence in a Bacillus pumilus plasmid: EcoRI endonuclease digestion of pPL576”, Journal of Bacteriology, July 1975, pg 377/379, Vol 123 (1). Furthermore this technology is known from the Handbook “Molecular Cloning: A Laboratory Manual”, ed. T. Maniatis, published by Cold Spring Harbor.

### BRIEF DESCRIPTION OF THE SEVERAL VIEWS OF THE DRAWINGS

Not Applicable.

### DETAILED DESCRIPTION OF THE INVENTION

The aim of the present invention is to generate an enzyme that is specific for one place in the genome.

In particular the aim of the present invention is to provide the possibility that the desired site in the genome can be chosen very specific. In case the chosen site in the genome (a regulating nucleotide sequence of a gene) is responsible for a disease, the present invention provides the possibility of shutting off this gene by targeting a specific enzyme to the regulating nucleotide sequence of the gene. The present invention provides this opportunity by means of the measures described in the characterizing part of claim 1.

Further advantageous embodiments are described in the dependent claims 2 to 3.

According to a further aspect the invention relates to the application of a vehicle, as mentioned before in combination with a SAINT-molecule, to transport a vehicle to a cell.

SAINT-molecules are already known, for example indicated as transport vehicle, and have been extensively described in the European Patent publication number EP-0 755 924-B1 and the U.S. Pat. No. 5,853, 694.

According to a preferred embodiment, the enzyme is coupled to the sequence recognizer by a spacer molecule. Thereby a spatial separation is generated between the sequence recognizing entity and the enzyme, as a result of which the enzyme activity is not inhibited when the vehicle is bound to the genome. In this way the enzyme is able to perform its function in the way the enzyme is normally used to.

In order to obtain a desired specificity for a favored place in the genome, preferable triple-helix forming units or oligonucleotides intercalating in the minor grooves of the DNA are used. That sort of sequence recognizers have a structure which conforms itself to the desired place in the genome only. As a result, the specificity becomes very high and, when these sequence recognizers are synthesized based on a known structure, they will be made unique to one place in the genome only. The technology is commonly known to a person skilled in the field and it is described in more detail in the article of F. Sanger, et al., “Use of DNA Polymerase 1 Primed by a synthetic Oligonucleotide to Determine the Sequence in Phage f1 DNA”, Proc. Nat. Acad. Sci. USA, Vol. 70, No. 4. pg 1209-1213, April 1973. Furthermore, this technique is described in the Handbook, “Molecular Cloning: A Laboratory Manual”, ed. T. Maniatis, published by Cold Spring Harbor.

Therefore it is preferred that the sequence recognizer has a DNA or PNA structure which is specific to the desired place in the genome. Such structures can be synthesized relatively easy.

According to another preferred embodiment, it is preferred that the desired enzyme has a low Km. In this way the enzyme will perform its action only if it is bound to the DNA. In case the enzyme is not fixed to the DNA, it will not be able to perform its activity. According to a preferred embodiment, it is preferred that the enzyme contains a so called “conformational switch.” Such a conformational switch takes care that the enzyme will not be active without the DNA being bound on the desired place. The reason for this is the otherwise inappropriately folded protein structure.

In order to facilitate a good transport of the vehicle to the cell, preferably the vehicle will be combined with a SAINT-molecule or a combination of SAINT-molecules. Optionally, additional compounds may be present with this.

It is particularly preferred that the SAINT-molecule interacts through hydrogen bonding. The SAINT-molecule enwraps the sequence recognizer and the enzyme that is covalently bound to the sequence recognizer. The SAINT-molecule as such is not bound to the vehicle (complex) but it just interacts through hydrogen bonding. This hydrogen bonding becomes looser when the complex contacts the cell contents after fusion with the cell membrane.

The DNA sequence, which is based on a 21 meric (this means a chain of 21 units) oligonucleotide, PNA or another entity, is specific for the human genome. A 21 meric appears only once. Notably, 4 to the power of 21 is much larger then the size of the human genome. As soon as TFO binds, the enzyme will fold around the DNA and performs its function. The oligonucleotide, however, will stay attached to the DNA, thereby assuring the enzyme can perform its function only once. At the moment the cell divides, the DNA will by “cleaned” from these uncommon proteins, the enzyme will be removed and be degraded through the usual ubiquitin route.

The invention has been essentially described above. Based on the description above and the attached conclusions, a person skilled in the art will easily be able to develop further embodiments, which, however, will fall within the scope of the present invention.

What is claimed is:

1. A vehicle for the transport of a DNA-modifying enzyme/molecule to the nucleus, said vehicle comprising the DNA-modifying enzyme/hybrid molecule combined with a SAINT-molecule or a combination of several entities thereof.

2. The vehicle according to claim 1, wherein the SAINT-molecule is bound to the DNA-modifying enzyme/hybrid molecule by means of hydrogen bonding.

3. Use of the SAINT-molecule in combination with the DNA-modifying enzyme/hybrid molecule according to claim 1, for the transport of said enzyme to the cell and to said nucleus thereof.
